SEQUENCE LISTING (3 sequences)

SEQ ID NO: 1 - DNA, 53 bp, synthetic construct
  source 1..53: mol_type = other DNA
ctgtctgtacgacgtcgcgggctccgataaatacggtcctgcaagcggttgtg

SEQ ID NO: 2 - DNA, 61 bp, synthetic construct
  source 1..61: mol_type = other DNA
gatccacaaccgcttgcaggaccgtatttatcggagcccgcgacgtcgtacagacagagct

SEQ ID NO: 3 - DNA, 2283 bp, synthetic construct
  source 1..2283: mol_type = other DNA
atgaagacttgggtaaaaatcgtatttggagttgccacctctgctgtgcttgccttattggtgatgtgcattgtcttacgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgaaaagtgtgaatgcttcaaattacggcttatcacctgatcggcaatttgtatatctagaaagtgattattcaaagctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaa

(19) United States
(12) Patent Application Publication
(10) Pub. No.: US 2025/0345311 A1
(43) Pub. Date: Nov. 13, 2025

(54) CONJUGATE OF POLYPEPTIDE AND SMALL MOLECULE TARGETING KRAS AND ANTI-CANCER APPLICATION THEREOF

(71) Applicant: Guangzhou Medical University, Guangzhou, Guangdong (CN)

(72) Inventors: Xiyong Yu, Guangzhou, Guangdong (CN); Ao Shen, Guangzhou, Guangdong (CN); Feng Yin, Guangzhou, Guangdong (CN); Lixin Zhao, Guangzhou, Guangdong (CN); Nanshan Zhong, Guangzhou, Guangdong (CN); Jishuo Chang, Guangzhou, Guangdong (CN)

(21) Appl. No.: 19/031,763

(22) Filed: Jan. 18, 2025

(30) Foreign Application Priority Data
May 10, 2024 (CN) 202410577046.3

(51) Int. Cl. A61K 31/415; A61K 47/64; C07K 7/54

(52) U.S. Cl. CPC A61K 31/415; A61K 47/64; C07K 7/54

(57) ABSTRACT

Provided is a conjugate of a polypeptide and a small molecule targeting KRAS and an anti-cancer application thereof. The conjugate of the polypeptide and small molecule is obtained by linking a cyclic peptide and a small molecule compound through chemical bonds. The structural formula of the cyclic peptide is as follows, and the small molecule compound is a small molecule inhibitor or its derivative that can inhibit GLI1 activity. This conjugate targets KRAS G12C and GLI1, exhibiting excellent anti-tumor activity and improving the susceptibility to drug-resistance in patients with tumors carrying KRAS mutations.

### CROSS-REFERENCE TO RELATED APPLICATION

This application claims priority of Chinese Patent Application No. 202410577046.3, filed on May 10, 2024, the entire contents of which are incorporated herein by reference.

### SEQUENCE LISTING

The present application contains a sequence listing which was filed electronically in XML format and is hereby incorporated by reference in its entirety. Besides, the XML copy is created on Jul. 7, 2025, is named “CONJUGATE OF POLYPEPTIDE AND SMALL MOLECULE TARGETING KRAS AND ANTI-CANCER APPLICATION THEREOF-Sequence Listing” and is 777.3 bytes in sizes.

### TECHNICAL FIELD

The present disclosure relates to the technical field of pharmaceuticals, in particular to a conjugate of a polypeptide and a small molecule targeting KRAS and an anti-cancer application thereof.

### BACKGROUND

Non-small cell lung cancer (NSCLC) is the most common type of lung cancer, accounting for approximately 80% of all lung cancers. The treatment of lung cancer patients mainly includes surgery, radiotherapy, chemotherapy, immunotherapy, and targeted therapy. At present, gene mutation site testing for EGFR, KRAS, BRAF, ALK, MET and other genes is the standard protocol for determining whether lung cancer patients should undergo targeted therapy. For patients with epidermal growth factor receptor (EGFR) sensitive mutations, the effective rate of using EGFR tyrosine kinase inhibitors (TKIs) reached 71.2%. KRAS mutation is the second most common gene mutation in NSCLC after EGFR mutation, occurring in approximately 25% of NSCLC cases, with the most common KRAS mutation being G12C mutation. KRAS G12C, as a driver mutation, activates signaling pathways such as MAPK and PI3K-AKT-mTOR, promoting cell proliferation and differentiation, and driving tumor development. At the same time, KRAS is also an important downstream regulatory gene of the EGFR signaling pathway, and KRAS G12C mutation can automatically activate without EGFR signaling, making NSCLC progress more rapidly and rendering EGFR-TKI targeted therapy ineffective. In addition to lung cancer, KRAS mutations are also common in pancreatic cancer, colorectal cancer, breast cancer and other malignant tumors. Therefore, developing targeted therapeutic medicines for KRAS mutations has broad application prospects.

For patients with KRAS G12C mutation, two KRAS G12C inhibitors, Sotorasib and Adagrasib, are currently approved for the treatment of advanced or metastatic NSCLC patients carrying KRAS G12C mutation who have received at least one systemic therapy in the past. Sotorasib also showed antitumor activity in pancreatic cancer and colorectal cancer. Although Sotorasib and Adagrasib have good therapeutic effects on cancer patients carrying KRAS G12C mutations in the early stages, they are highly susceptible to developing drug-resistance. At present, there is no good treatment plan or medication in clinical practice that can significantly prolong the survival of patients who develop drug-resistance. Single target medicine therapy is no longer able to cope with the increasing number of mutation types in lung cancer patients in clinical practice. Therefore, it is urgent to search for new lung cancer medicine targets to conduct multi-target intervention therapy for KRAS G12C mutant lung cancer patients, in order to achieve better therapeutic effects.

The activation of the Hedgehog signaling pathway plays an important role in the KRAS mutation process, and the activation of the Hedgehog pathway is one of the necessary conditions for maintaining the growth of NSCLC cells carrying KRAS mutations. Glioma-associated oncogene homolog 1 (GLI1) is the terminal effector of the Hedgehog pathway. GLI1 has been found to play a decisive role in the occurrence and development of various cancers, including lung cancer. Overexpression of GLI1 is positively correlated with the exacerbation of tumor malignancy. The applicant has previously reported that inhibiting GLI1 can suppress angiogenesis in lung cancer, and has also found that GLI1 promotes metastasis and invasion of NSCLC tumor cells by regulating Snail. Meanwhile, GLI1 also regulates the expression of various genes related to tumor drug-resistance, such as SOX2, OCT4, AXL, etc.

### SUMMARY

In view of above, the present disclosure has developed a conjugate of a polypeptide and a small molecule, which can specifically target KRAS G12C and release GLI1 small molecule inhibitors that can kill or inhibit cancer cell growth at the tumor site. The conjugate intervenes against both KRAS G12C and GLI1 target spots and has good anti-tumor activity.

The present disclosure includes the following technical solutions.

In first aspect, the present disclosure provides a conjugate of a polypeptide and a small molecule, which is obtained by linking a cyclic peptide and a small molecule compound through a chemical bond.

The structural formula of the cyclic peptide is:

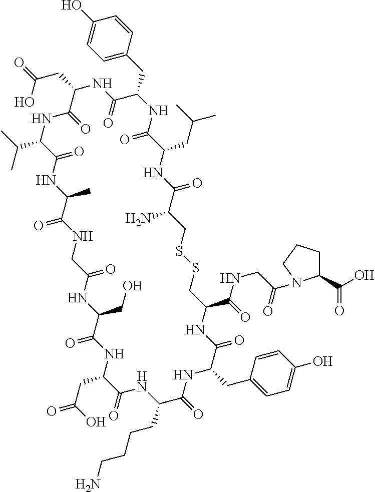 [SMILES: CC(C)C[C@@H]1NC(=O)[C@@H](N)CSSC[C@@H](C(=O)NCC(=O)N2CCC[C@H]2C(=O)O)NC(=O)[C@H](Cc2ccc(O)cc2)NC(=O)[C@H](CCCCN)NC(=O)[C@H](CC(=O)O)NC(=O)[C@H](CO)NC(=O)CNC(=O)[C@H](C)NC(=O)[C@H](C(C)C)NC(=O)[C@H](CC(=O)O)NC(=O)[C@H](Cc2ccc(O)cc2)NC1=O]

The small molecule compound is a small molecule inhibitor or its derivatives being capable of inhibiting GLI1 activity.

In some embodiments, the small molecule compound is selected from the following compounds:

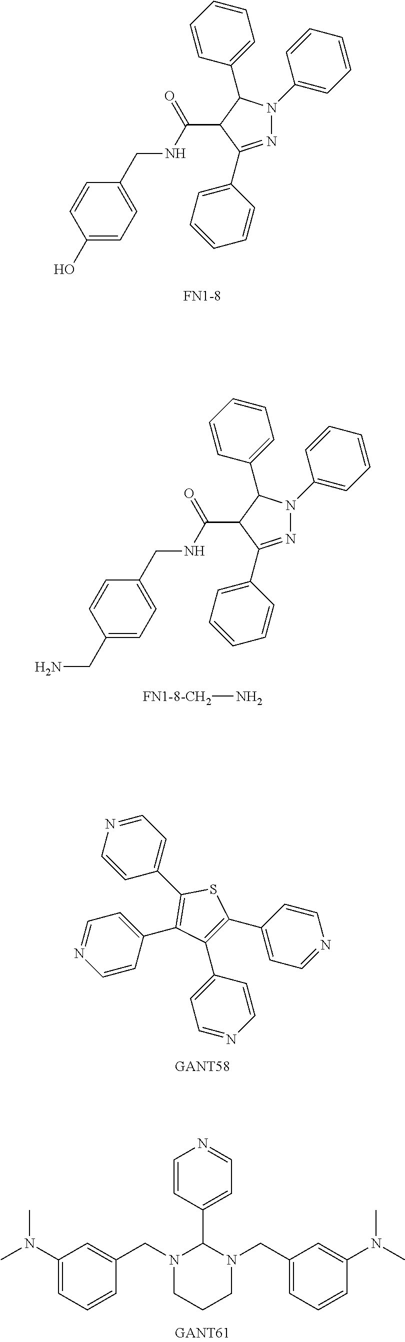

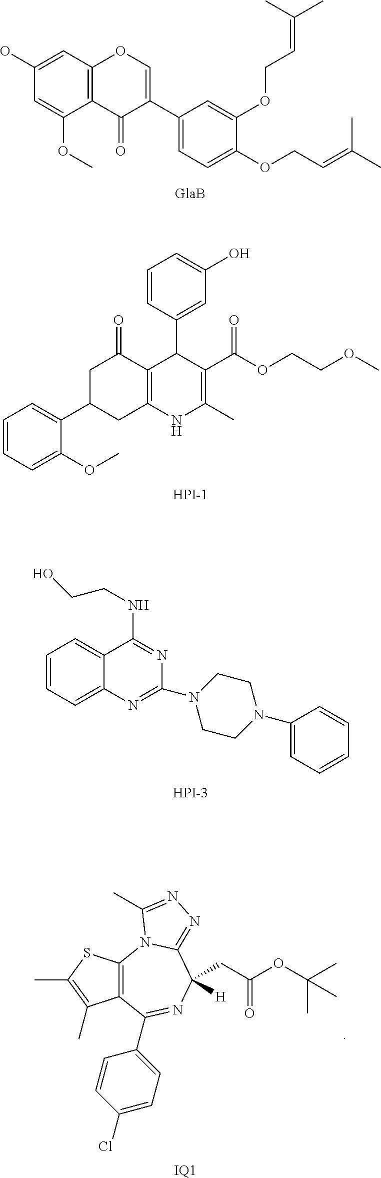

In some of these embodiments, the carboxyl group at a C-terminal of the cyclic peptide is connected to the small molecule compound through an amide bond or an ester bond.

In some of these embodiments, the structural formula of the conjugate of the polypeptide and the small molecule is:

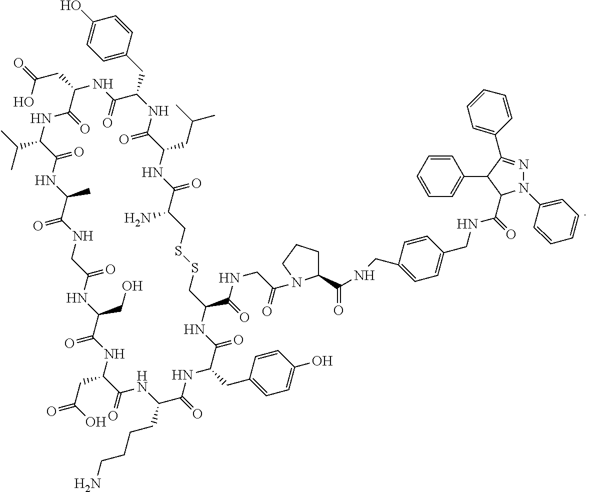 [SMILES: CC(C)C[C@@H]1NC(=O)[C@@H](N)CSSC[C@@H](C(=O)NCC(=O)N2CCC[C@H]2C(=O)NCc2ccc(CNC(=O)C3C(c4ccccc4)C(c4ccccc4)=NN3c3ccccc3)cc2)NC(=O)[C@H](Cc2ccc(O)cc2)NC(=O)[C@H](CCCCN)NC(=O)[C@H](CC(=O)O)NC(=O)[C@H](CO)NC(=O)CNC(=O)[C@H](C)NC(=O)[C@H](C(C)C)NC(=O)[C@H](CC(=O)O)NC(=O)[C@H](Cc2ccc(O)cc2)NC1=O]

The second aspect, the present disclosure further provides the application of the conjugate of the polypeptide and the small molecule in the preparation of a medicine for preventing and/or treatment of tumors.

In some embodiments, the tumors are lung cancer, pancreatic cancer, colorectal cancer, and breast cancer.

In some embodiments, the tumor is a tumor carrying KRAS mutation, wherein the KRAS mutation is a KRAS G12C mutation, namely, the tumor is further preferred to be a tumor carrying KRAS G12C mutation.

In some embodiments, the tumor is a tumor overexpressing GLI1. The tumor described in the present disclosure can be a tumor carrying KRAS G12C mutation, a tumor overexpressing GLI1, or a tumor carrying KRAS G12C mutation and overexpressing GLI1.

In some embodiments, the tumor is non-small cell lung cancer. Further, the non-small cell lung cancer overexpresses GLI1, or the non-small cell lung cancer carries a KRAS G12C mutation, or the non-small cell lung cancer carries a KRAS G12C mutation while overexpressing GLI1.

The third aspect, the present disclosure further provides a medicine for preventing and/or treating tumors, wherein the medicine is prepared from an active ingredient and a pharmaceutically acceptable excipient. The active ingredients include the conjugate of the polypeptide and the small molecule, and/or FN1-8 methylamino derivatives or their pharmaceutically acceptable salts according to the present disclosure.

The present disclosure provides a conjugate of a polypeptide and a small molecule with novel structure, which obtains by linking a CLYDVAGSDKYCGP cyclic peptide and a GLI1 small molecule inhibitor through a chemical bond. The cyclic peptide in the conjugate can specifically target the KRAS G12C protein. The GLI1 small molecule inhibitor can inhibit the activity of GLI1 protein, which can suppress the proliferation of cancer cells and also cut off the downstream activated oncogenic pathway after KRAS mutation. This conjugate targets KRAS G12C and GLI1, exhibiting excellent anti-tumor activity and improving the susceptibility of cancer patients carrying KRAS mutations to drug-resistance.

In addition, the dipeptide linker GP in the cyclic peptide is a substrate recognition sequence for fibroblast activation protein (FAPα), and FAPα is highly expressed in tumor associated fibroblasts. Therefore, the conjugate of the present disclosure is only cleaved and releases GLI1 small molecule inhibitors at the tumor site, which has tumor responsiveness and reduces systemic toxicity of the medicine. It can greatly reduce the toxic side effects of the medicine and improve its medication safety.

The present disclosure couples the specific cyclic peptide with the small molecule inhibitor, which not only improves the disadvantages of strong water solubility and low bioavailability of peptides, but also reduces the non-selective toxicity of small molecule medicines such as FN1-8, improves medicine safety, and the modified cyclic peptide has better in vivo stability compared to the linear peptide LYDVAGSDKY. Therefore, the conjugate of the present disclosure is a very promising therapeutic agent for KRAS-mutant tumors, offering dual-targeting capabilities, the ability to overcome drug-resistance, and high efficacy in treating lung cancer and other malignant tumors carrying KRAS G12C mutations.

### BRIEF DESCRIPTION OF THE DRAWINGS

FIG. 1 shows the HPLC chromatogram of LYDVAGSDKY linear peptide.

FIG. 2 shows the mass spectrum of LYDVAGSDKY linear peptide.

FIG. 3 shows the HPLC chromatogram of CLYDVAGSDKYC cyclic peptide.

FIG. 4 shows the mass spectrum of CLYDVAGSDKYC cyclic peptide.

FIG. 5 shows the HPLC chromatogram of CLYDVAGSDKYCGP cyclic peptide.

FIG. 6 shows the mass spectrum of CLYDVAGSDKYCGP cyclic peptide.

FIG. 7 shows the hydrogen spectrum of FN1-8 methylamino derivative (FN1-8-CH2-NH2).

FIG. 8 shows the HPLC chromatogram of the conjugate HGPF.

FIG. 9 shows the mass spectrum of the conjugate HGPF.

FIG. 10 shows the serum stability HPLC chromatogram of LYDVAGSDKY linear peptide.

FIG. 11 shows the serum stability HPLC chromatogram of CLYDVAGSDKYC cyclic peptide.

FIG. 12 shows the stability line chart of linear and cyclic peptides.

FIG. 13 shows the HPLC chromatogram of the conjugate HGP-FITC.

FIG. 14 shows the mass spectrum of the conjugate HGP-FITC.

FIG. 15 shows the uptake of HGP-FITC at different concentrations by lung cancer cells carrying KRAS G12C mutation.

FIG. 16 shows the specificity analysis of HGP-FITC targeting KRAS G12C.

FIG. 17 shows the BRET experimental analysis of drug response to FAPα.

FIG. 18 shows the cytotoxicity of drugs on tumor cells of CCK8 experiment analysis.

FIG. 19 shows the scratch test analysis of the inhibitory effect of drugs on tumor cell proliferation and migration.

### DETAILED DESCRIPTION OF THE EMBODIMENTS

The experimental methods in the following embodiments of the present disclosure that do not specify specific conditions are usually carried out under conventional conditions or conditions recommended by the manufacturer. The various commonly used chemical reagents used in the embodiments are all commercially available products.

Unless otherwise defined, all technical and scientific terms used in the present disclosure have the same meanings as those commonly understood by those skilled in the art relating to the present disclosure. The terms used in the specification of the present disclosure are only for the purpose of describing specific embodiments and are not intended to limit the present disclosure.

The terms “including” and “having” in the present disclosure, as well as any variations thereof, are intended to cover non exclusive inclusion. For example, a process, method, apparatus, product, or equipment that includes a series of steps is not limited to the listed steps or modules, but may optionally also include steps that are not listed, or may optionally include other steps inherent to these processes, methods, products, or equipment.

The term “a plurality of” mentioned in the present disclosure refers to two or more. “And/or” describes the association relationship between related objects, indicating that there can be three types of relationships, for example, A and/or B, which can represent: A exists alone, A and B exist simultaneously, and B exists alone. The character “/” generally indicates that the associated objects are an “or” relationship.

In one embodiment of the present disclosure, a conjugate of a polypeptide and a small molecule is provided. The conjugate is obtained by linking a cyclic peptide and a small molecule compound through a chemical bond.

The structural formula of the cyclic peptide is:

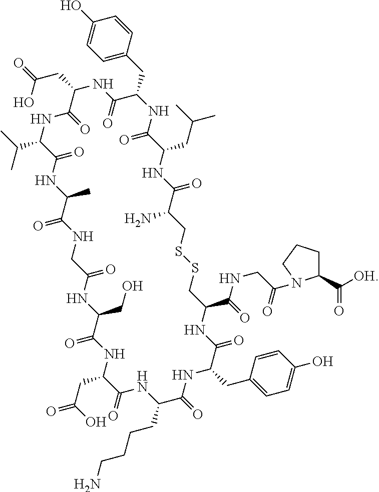 [SMILES: CC(C)C[C@@H]1NC(=O)[C@@H](N)CSSC[C@@H](C(=O)NCC(=O)N2CCC[C@H]2C(=O)O)NC(=O)[C@H](Cc2ccc(O)cc2)NC(=O)[C@H](CCCCN)NC(=O)[C@H](CC(=O)O)NC(=O)[C@H](CO)NC(=O)CNC(=O)[C@H](C)NC(=O)[C@H](C(C)C)NC(=O)[C@H](CC(=O)O)NC(=O)[C@H](Cc2ccc(O)cc2)NC1=O]

The small molecule compound is a small molecule inhibitor or its derivatives that are capable of inhibiting GLI1 activity.

In another embodiment of the present disclosure, a medicine for preventing and/or treating tumors is provided, which is prepared from an active ingredient and a pharmaceutically acceptable excipient, wherein the active ingredient includes the conjugate of the polypeptide and the small molecule of the present disclosure, and/or FN1-8 methylamino derivatives or their pharmaceutically acceptable salts as described in the present disclosure.

The medicine for preventing and/or treating tumors provided by the present disclosure includes active ingredients within a safe and effective dosage range (i.e., the conjugate of the polypeptide and small molecule of the present disclosure, and/or the FN1-8 methylamino derivatives or their pharmaceutically acceptable salt of the present disclosure), as well as pharmaceutically acceptable excipients. When administering medicines, a safe and effective amount of the conjugate or compound of the present disclosure is applied to a mammal (such as a human) in need of treatment, where the dosage at the time of administration is the pharmaceutically acceptable effective dosage. Of course, the specific dosage should also consider factors such as the route of administration and the health condition of the patient, which are within the skill range of skilled physicians.

Wherein, “safe and effective dosage” refers to the amount of active ingredient that is sufficient to significantly improve the condition without causing serious side effects. “Pharmaceutical acceptable excipient” refers to one or more solid or liquid fillers or gel substances with compatibility, which are suitable for human use and must have sufficient purity and low toxicity. “Compatibility” here refers to the ability of each component in the composition to blend with the active ingredient of the present disclosure and their interactions without significantly reducing the efficacy of the active ingredient.

Examples of pharmaceutically acceptable excipients include cellulose and its derivatives (such as sodium carboxymethylcellulose, sodium ethylcellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agents (such as sodium dodecyl sulfate), coloring agents, seasonings, stabilizers, antioxidants, preservatives, pyrogen-free water, etc.

The administration method of the active ingredient or pharmaceutical composition of the present disclosure is not particularly limited, and representative administration methods include (but are not limited to) oral administration, intratumoral administration, rectal administration, parenteral administration (intravenous, intramuscular or subcutaneous), etc.

Solid dosage forms for oral administration include capsules, tablets, pills, powders, and granules.

In these solid dosage forms, the active ingredient is mixed with at least one conventional inert excipient (or carrier), such as sodium citrate or dicalcium phosphate, or mixed with the following ingredients:
(a) Fillers or compatibilizers, such as starch, lactose, sucrose, glucose, mannitol, and silicic acid;
(b) Adhesives, such as hydroxymethyl cellulose, alginate, gelatin, polyvinylpyrrolidone, sucrose, and arabic gum;
(c) Moisturizing agents, such as glycerin;
(d) Disintegrants, such as agar, calcium carbonate, potato starch or cassava starch, alginic acid, certain complex silicates, and sodium carbonate;
(e) Retardants, such as paraffin wax;
(f) Absorption accelerators, such as quaternary amine compound;
(g) Wetting agents, such as cetyl alcohol and glycerol monostearate;
(h) Adsorbents, such as kaolin;
(i) Lubricants, such as talc, calcium stearate, magnesium stearate, solid polyethylene glycol, sodium dodecyl sulfate, or mixtures thereof. Capsules, tablets, and pills may also contain buffering agents.

The solid dosage forms can also be prepared using coatings and shell materials, such as casings and other materials known in the art. They may contain opaque agents, and the release of active ingredients in this composition can be delayed in a certain part of the digestive tract. Examples of usable embedding components are polymeric substances and wax based substances.

Liquid dosage forms for oral administration include pharmaceutically acceptable emulsions, solutions, suspensions, syrups or tinctures. In addition to the active ingredients, liquid dosage forms may include inert diluents commonly used in the field, such as water or other solvents, solubilizers, and emulsifiers, such as ethanol, isopropanol, ethyl carbonate, ethyl acetate, propylene glycol, 1,3-butanediol, dimethylformamide, as well as oils, particularly cottonseed oil, peanut oil, corn germ oil, olive oil, castor oil, and sesame oil, or mixtures of these substances. In addition to these inert diluents, the composition may also contain adjuvants such as wetting agents, emulsifiers and suspensions, sweeteners, taste corrector, and flavorings.

In addition to active ingredients, suspensions may contain suspending agents such as ethoxylated isooctadecanol, polyoxyethylene sorbitol and dehydrated sorbitol esters, microcrystalline cellulose, aluminium methoxide and agar, or mixtures of these substances.

Compositions for parenteral injection may include physiologically acceptable sterile aqueous or anhydrous solutions, dispersions, suspensions or emulsion, and sterile powders for re-dissolution into sterile injectable solutions or dispersions. Suitable aqueous and non-aqueous carriers, diluents, solvents, or excipients include water, ethanol, polyols, and their suitable mixtures.

The conjugate or compound of the present disclosure can be administered alone or in combination with other medicines known for treating or improving similar conditions.

The following are specific embodiments, and all reagents, materials, and raw materials in the following embodiments can be obtained through commercial channels.

### Embodiment 1: Synthesis of LYDVAGSDKY Linear Peptide and CLYDVAGSDKY Cyclic Peptide

Firstly, LYDVAGSDKY and CLYDVAGSDKY linear peptides were synthesized using solid-phase synthesis method, as follows.

### 1.1 Treatment of Solvents

Soak DMF, methanol in G3 well molecular sieve overnight to remove impurities and water before use.

### 1.2 Adequate Swelling of Resin

Weigh 2.0 g of blank 2-chlorotrityl chloride resin (2-CTC Resin) into a clean and dry reaction tube, add 15 mL of DMF, and activate at room temperature for about 30 minutes.

### 1.3 Connecting to the First Amino Acid at the N-Terminal

At room temperature, the solvent in the swollen resin was filtered out through a sand core, and 1 mmol of 5-fold molar excess of the N-terminal first amino acid Fmoc-Leu-OH (when synthesizing LYDVAGSDKY) or Fmoc-Cys0OH (when synthesizing CLYDVAGSDKYC), 5-fold molar excess of DMAP, 5-fold molar excess of DIC were added. DMF was added as the solvent, and the reaction was carried out at room temperature for 3 hours. After the reaction was complete, wash 4-6 times with DMF, each time with 5-6 mL. Add 2 ml of pyridine and acetic anhydride in a volume ratio of 1:1 and react for 30 minutes. After the reaction was complete, wash 4-6 times with DMF, 5-6 mL each time (to block the active sites on the unreacted empty resin).

### 1.4 Removal of Fmoc Protective Group

Filter out the solvent from the previous step, add 10 mL of 20% piperidine DMF solution to the resin, stir by blowing of N2 for 10 minutes, filter out the solution, then add 10 mL of 20% piperidine DMF solution, stir by blowing of N2 for 5 minutes, and filter out the solution. Repeat this process twice, wash with DMF 4 times and methanol 2 times, each time 5-6 mL.

### 1.5 Ninhydrin Detection Removal Effect

Take out a small amount of resin, wash it three times with methanol, then add one drops of ninhydrin, KCN, phenol solution respectively, heat at 105° C.-110° C. for 5 minutes, turning dark blue indicates a positive reaction, it indicates complete removal, and the next step of the reaction can proceed; if colorless, it indicates that the protective group has not been completely removed, and the above removal operation needs to be repeated.

### 1.6 Removal of the Second Amino Acid and Fmoc Protective Group

Weigh 3-fold molar excess of the N-terminal second amino acid Fmoc-Try-OH (when synthesizing LYDVAGSDKY) or Fmoc-Leu-OH (when synthesizing CLYDVAGSDKYC), 3-fold molar excess of HBTU, and 3-fold molar excess of HOBT into the deprotection reaction system in the previous step, add an appropriate amount of DMF solution to completely dissolve them, then add 10-fold the molar excess of pure DIEA, react at room temperature for 40 minutes, and then wash 4-6 times with DMF, each time 5-6 mL. Take a small amount of resin and detect it with ninhydrin detection reagent, if the color appears colorless, then add 10 mL of 20% piperidine DMF solution to remove Fmoc, and perform two washes, 10 min and 5 min respectively. Afterwards, wash with DMF four times and methanol two times, each time 5-6 mL. Take out a small amount of resin and test it with the ninhydrin detection reagent. If the color turns blue, proceed to the next step of the reaction.

### 1.7 Removal of Residual Amino Acids and Fmoc Protective Group

Similarly, repeat step 1.6 until the last amino acid at the C-terminal of the peptide was synthesized, remove the Fmoc protective group, and detect NH2 with ninhydrin, if the color appears blue, proceed to the next step of the reaction.

### 1.8 Connecting Boc Protective Group

Add 2 times the equivalent of di-tert-butyl dicarbonate (Boc) 20 and react at room temperature for 2 hours using DMF as the solvent. Drain the solvent and wash the resin twice with methanol, dichloromethane, and DMF in sequence. Use ninhydrin for coloration test, if the color appears colorless, indicating that the Boc connection was complete, then drain the resin again.

### 1.9 Resin Detachment and Peptide Separation

Using trifluoroacetic acid cutting solution (1% TFA:2% TIS:2% EDT:95% H2O), the resin obtained in step 1.8 was conducted fully protected cutting twice, each time for 10 minutes, and the cutting solution was collected twice and freeze-dried to obtain fully protected linear peptides LYDVAGSDKY and CLYDVAGSDKYC.

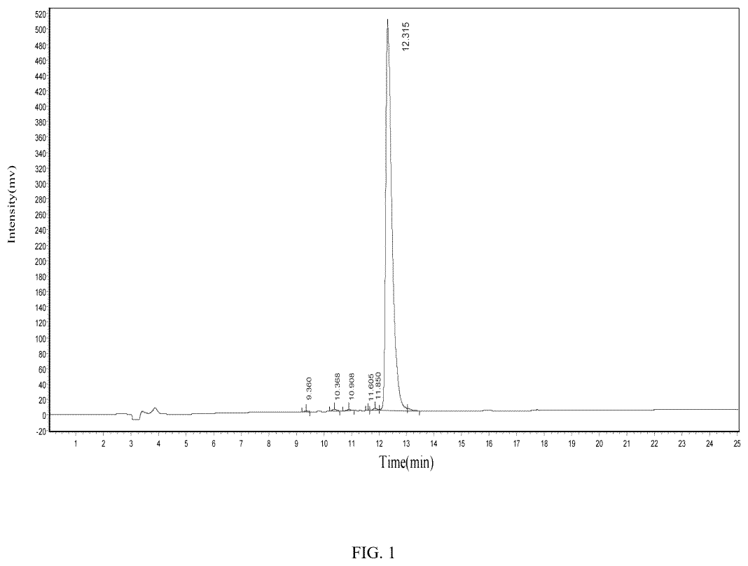

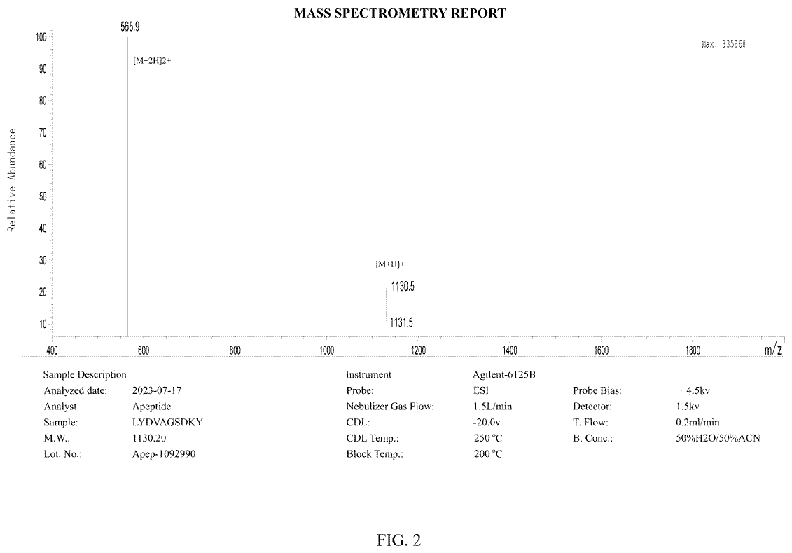

The HPLC detection results of the linear peptide LYDVAGSDKY are shown in FIG. 1, and its mass spectrum is shown in FIG. 2. The above results demonstrate the acquisition of LYDVAGSDKY linear peptide.

### 1.10 Cyclization of CLYDVAGSDKYC Linear Peptide

Add the CLYDVAGSDKYC linear peptide obtained in the previous step to a 5 mM NH4HCO3 aqueous solution, adjust the peptide concentration to 0.1 mg/mL, and then add a final concentration of 5% hydrogen peroxide. After 1 hour of reaction, disulfide bonds will form between two sulfhydryl groups of cysteine in the linear peptide. After freeze-drying, CLYDVAGSDKYC fully protected cyclic peptide can be obtained.

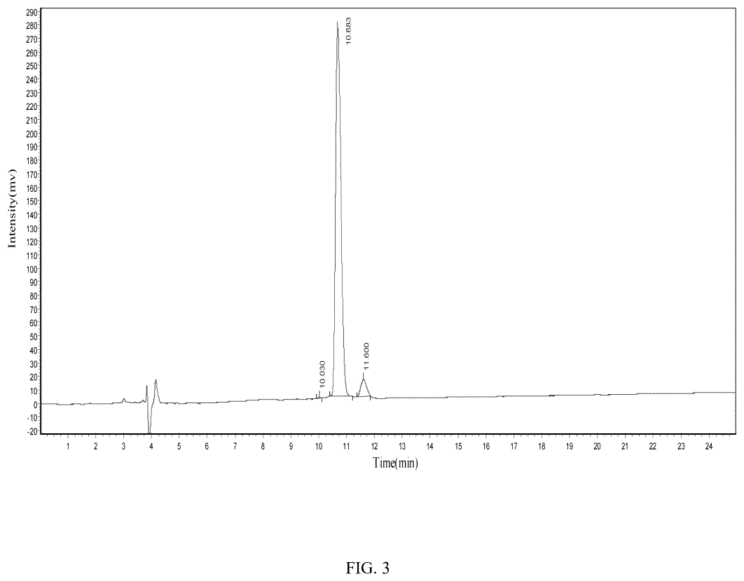

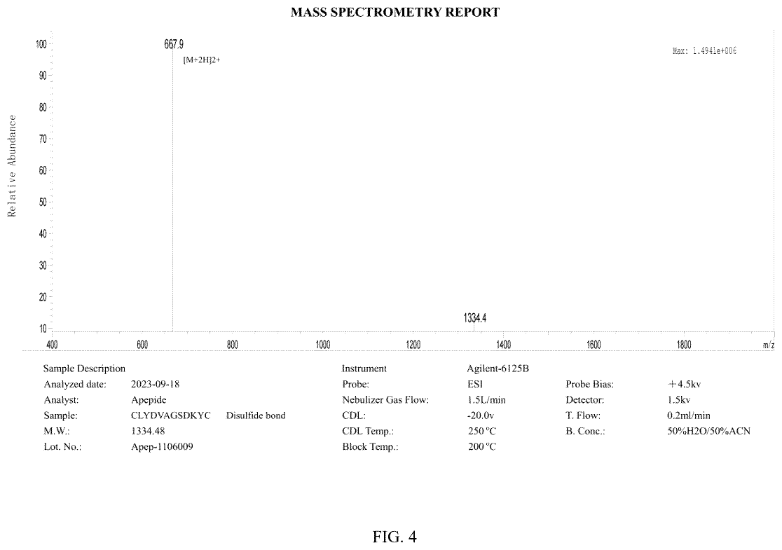

The HPLC detection results of CLYDVAGSDKYC cyclic peptide are shown in FIG. 3, and its mass spectrum is shown in FIG. 4. The above results demonstrate the acquisition of CLYDVAGSDKYC cyclic peptide. The sequence of the CLYDVAGSDKYC cyclic peptide is shown in SEQ ID NO: 5, and it is Cys Leu Tyr Asp Val Ala Gly Ser Asp Lys Tyr Cys.

### Embodiment 2: Synthesis of Cyclic Peptide with GP Linker (CLYDVAGSDKYCGP)

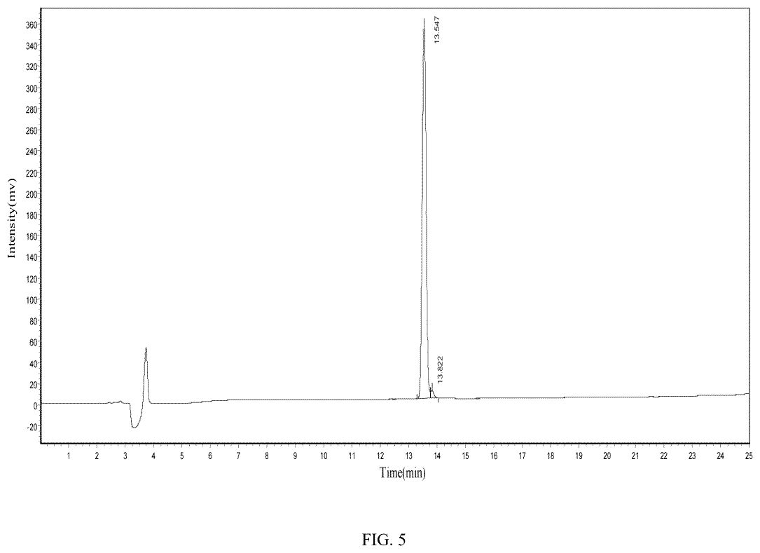

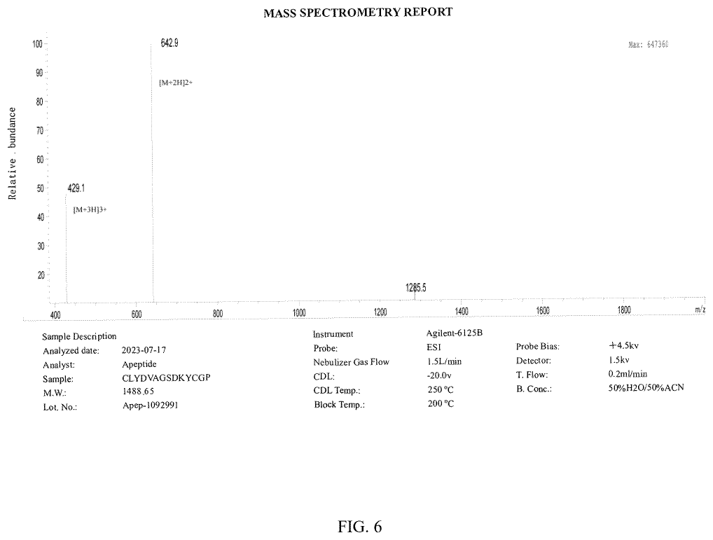

Following the method described in Embodiment 1, CLYDVAGSDKYCGP linear peptide was synthesized first, followed by cyclization to obtain the CLYDVAGSDKYCGP cyclic peptide. The structure of the product was identified using techniques such as HPLC (as shown in FIG. 5) and mass spectrometry (as shown in FIG. 6), and the results showed that the cyclic peptide CLYDVAGSDKYCGP was successfully synthesized, with the following structural formula:

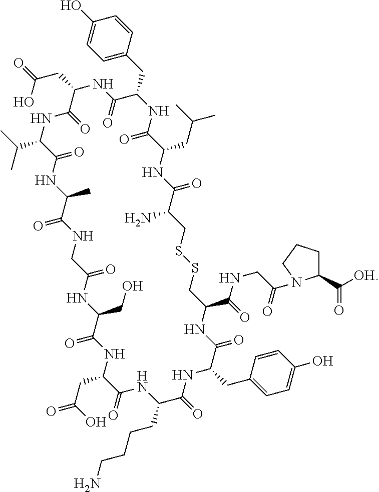 [SMILES: CC(C)C[C@@H]1NC(=O)[C@@H](N)CSSC[C@@H](C(=O)NCC(=O)N2CCC[C@H]2C(=O)O)NC(=O)[C@H](Cc2ccc(O)cc2)NC(=O)[C@H](CCCCN)NC(=O)[C@H](CC(=O)O)NC(=O)[C@H](CO)NC(=O)CNC(=O)[C@H](C)NC(=O)[C@H](C(C)C)NC(=O)[C@H](CC(=O)O)NC(=O)[C@H](Cc2ccc(O)cc2)NC1=O]

### Embodiment 3: Synthesis of FN1-8 Methylamino Derivative (FN1-8-CH2-NH2)

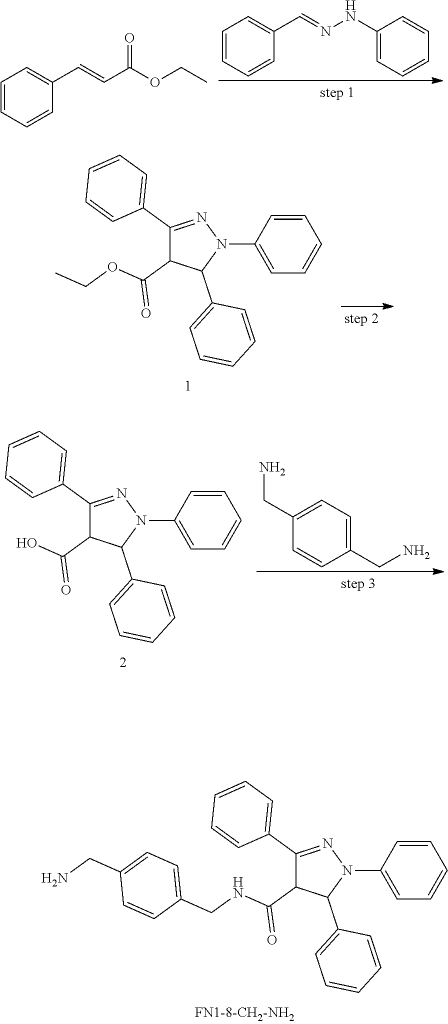

3.1 Heat a mixture of ethyl cinnamate (0.52 ml), benzaldehyde phenylhydrazine (600 mg), chloramine-T trihydrate (844 mg), and methanol (5 mL) under reflux for 24 hours under nitrogen protection. Dilute the reaction mixture with 400 ml of a 1:1 mixture of ethyl acetate and n-hexane, filter through a sand core funnel, mix with an appropriate amount of silica gel, then evaporate and spin dry using a rotary evaporator. The residue was separated and purified by column chromatography (petroleum ether:ethyl acetate=15:1) to obtain compound 1 (650 mg) with a yield of 67%.

3.2 Mix compound 1 (2.00 g) evenly with methanol (4 ml), 1,4-dioxane (2 ml), and 10% sodium chloride aqueous solution (2 ml), and react at room temperature for 1 hour. After diluting the reaction solution with 30 ml of water, the reaction product was washed with a 3:10 mixture solution of ethyl acetate:n-hexane, and then separated using a separatory funnel. The aqueous phase was acidified with disodium chromotropate dihydrate, and then extracted with ethyl acetate. Wash the extract twice with water and brine, and finally dry the extract with anhydrous sodium sulfate. After stirring the extract with an appropriate amount of silica gel, it was evaporated and dried using a vacuum rotary evaporator. The residue was purified by column chromatography (petroleum ether:ethyl acetate=15:1) to obtain compound 2 (410 mg) with a yield of 68%.

3.3 Mix compound 2 (1.5 g) with THF (20 ml), DIEA (1.70 mg), T4P (4.70 g), and 1,4-bis(aminomethyl)benzene (830 mg), and react at room temperature for 1 hour; wash the reaction solution with salt water, saturated sodium sulfate aqueous solution, and water; separate the solution using a separatory funnel, dry the organic phase with anhydrous sodium sulfate, then stir it evenly with an appropriate amount of silica gel, and evaporate the mixture using a vacuum rotary evaporator. The residue was separated and purified by column chromatography, and finally to obtain FN1-8-CH2-NH2 (357 mg), a methylamino derivative of FN1-8, with a yield of 65% and a purity greater than 97%.

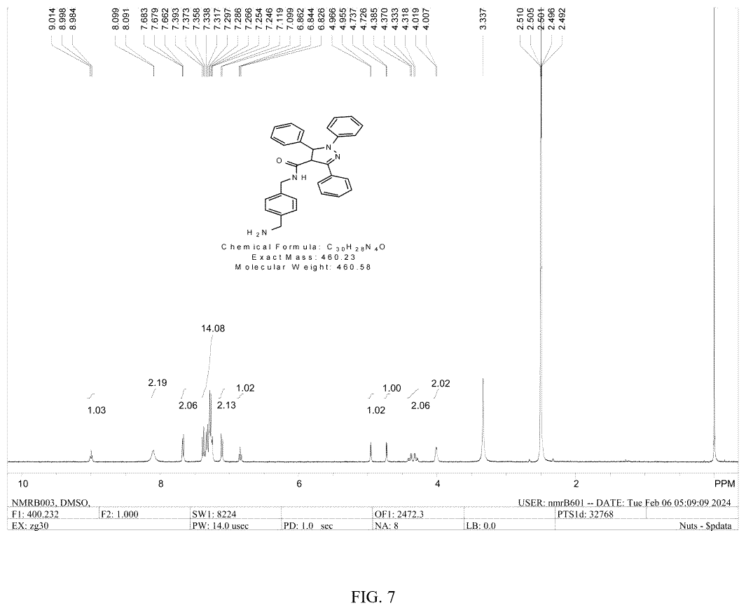

The nuclear magnetic hydrogen spectrum of the methylamino derivative of FN1-8-CH2-NH2 is shown in FIG. 7.

### Embodiment 4: Synthesis of Conjugate of Cyclic Peptide with FN1-8 Methylamino Derivative (HGPF)

Dissolve the CLYDVAGSDKYCGP cyclic peptide obtained in Embodiment 2 in 15 ml of anhydrous pyridine and cool to −15° C., add 1 mL of POCl3 to the solution, then gradually dropwise add anhydrous dichloromethane solution which dissolves the FN1-8 methylamino derivative obtained in Embodiment 3 (100 mg of FN1-8-CH2-NH2 dissolved in 200 ml of dichloromethane) under −15° C. condition. React for 2 hours and obtain crude product after rotary drying. Add cutting liquid (95% TFA, 2% TIS, 2% EDT, 1% H2O) and react for 2 hours to obtain the target product HGPF 11.45 mg.

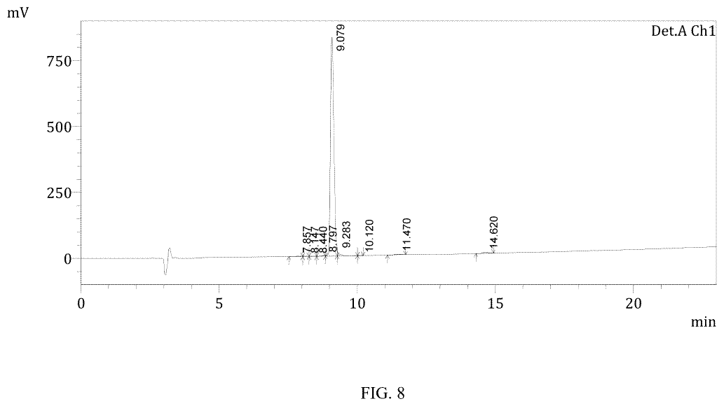

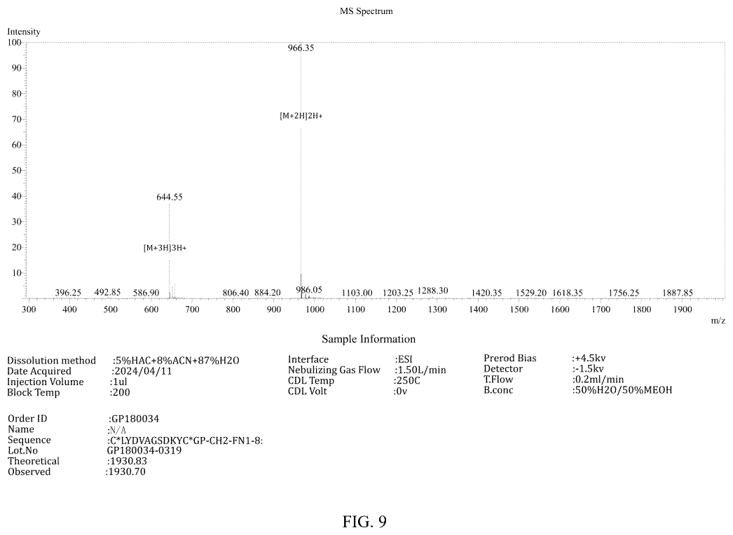

After identification by HPLC (FIG. 8) and mass spectrometry (FIG. 9), it was confirmed that the conjugate HGPF was successfully obtained by linking the cyclic peptide with FN1-8 methylamino derivative via GP dipeptide linker. Its structure is as follows:

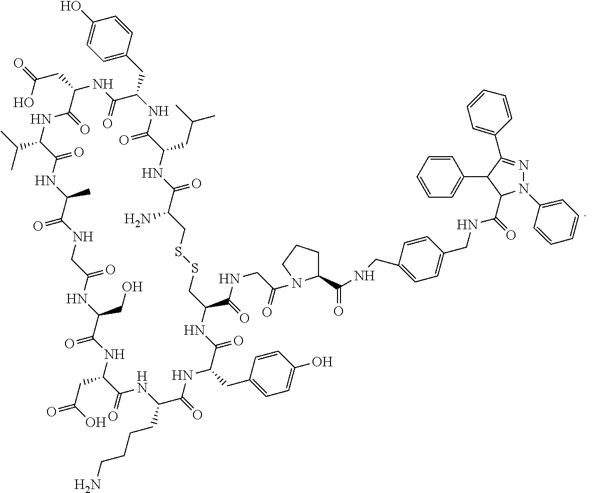 [SMILES: CC(C)C[C@@H]1NC(=O)[C@@H](N)CSSC[C@@H](C(=O)NCC(=O)N2CCC[C@H]2C(=O)NCc2ccc(CNC(=O)C3C(c4ccccc4)C(c4ccccc4)=NN3c3ccccc3)cc2)NC(=O)[C@H](Cc2ccc(O)cc2)NC(=O)[C@H](CCCCN)NC(=O)[C@H](CC(=O)O)NC(=O)[C@H](CO)NC(=O)CNC(=O)[C@H](C)NC(=O)[C@H](C(C)C)NC(=O)[C@H](CC(=O)O)NC(=O)[C@H](Cc2ccc(O)cc2)NC1=O]

### Embodiment 5: Stability Testing of Polypeptide

### 5.1 Serum Preparation

Healthy C57 male mice (18-24 g) were subjected to orbital vein blood collection to collect fresh blood and placed in a 1.5 ml centrifuge tube. The tube was left to stand for 1 hour until the blood coagulated, then centrifuged at 3000 g and 4° C. for 15 minutes. The supernatant was collected and used for peptide stability studies.

### 5.2 Incubation Experiment

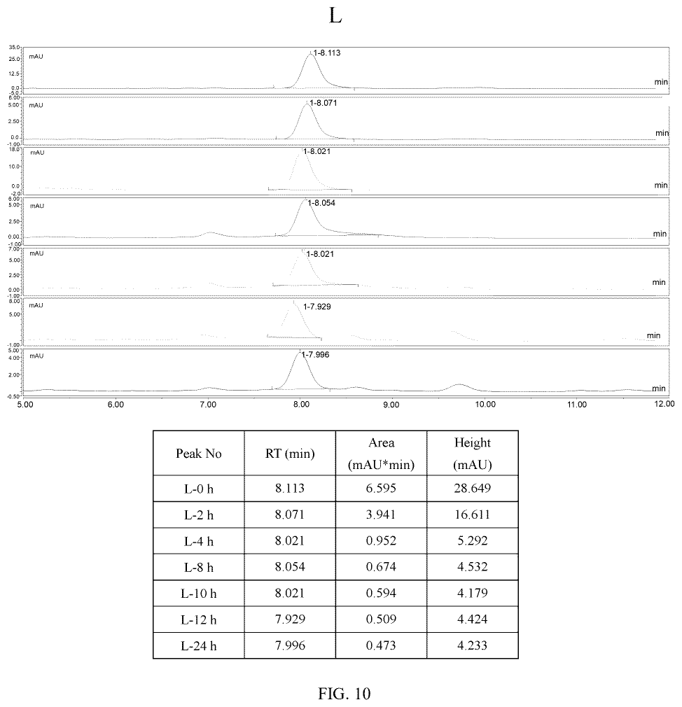

Prepare 10 mM stock solutions of LYDVAGSDKY linear peptide and CLYDVAGSDKYC cyclic peptide with physiological saline, respectively. Take 40 μl of the stock solution to add to 360 μl of mouse serum and 360 μl of pH 7.4 buffer solution (polypeptide drug to serum/PBS ratio is 1:9). Immediately vortex and mix well and incubate in a 37° C. constant temperature incubator. At time points 0, 2 h, 4 h, 8 h, 10 h, 12 h, and 24 h, take 40 μl samples into 1.5 ml pre-cooled centrifuge tubes, and add 40 μl of ice acetonitrile to the 1.5 ml pre-cooled centrifuge tubes to inactivate the enzyme, respectively. After 5 minutes of ice bath, add 20 μl of 0.5% ice acetic acid/water to ensure that the enzymatic hydrolysis process was stopped, terminate the reaction and vortex for mixing. The sample was centrifuged at 4° C. and 15000 g for 30 minutes. The supernatant was taken and diluted 5 times with deionized water. Then, HPLC was immediately used to detect the peak time, peak area, and other indicators of the linear peptide and the cyclic peptide (as shown in FIG. 10 and FIG. 11).

### 5.3 Result Analysis

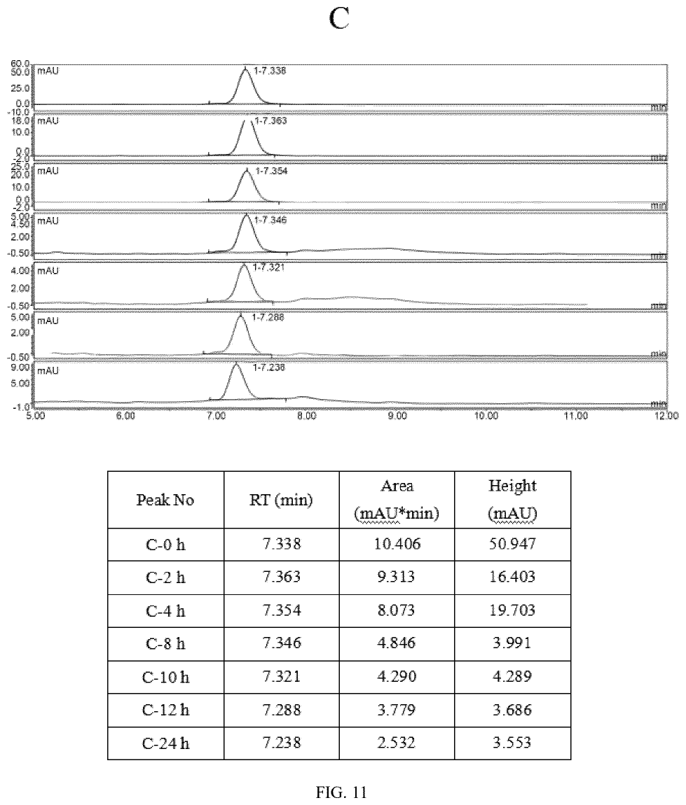

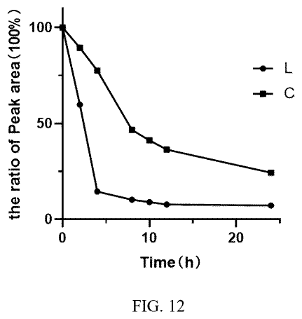

Using a peptide content of 0 hours as a standard, it can be observed that under the same incubation time, the degradation rate of linear peptides is significantly faster than that of cyclic peptides, and the stability of cyclic peptides is significantly better than that of linear peptides (as shown in FIG. 12).

### Embodiment 6: Targeting Ability of Cyclic Peptide Conjugates to KRAS G12C

### 6.1 Synthesis of HGP-FITC

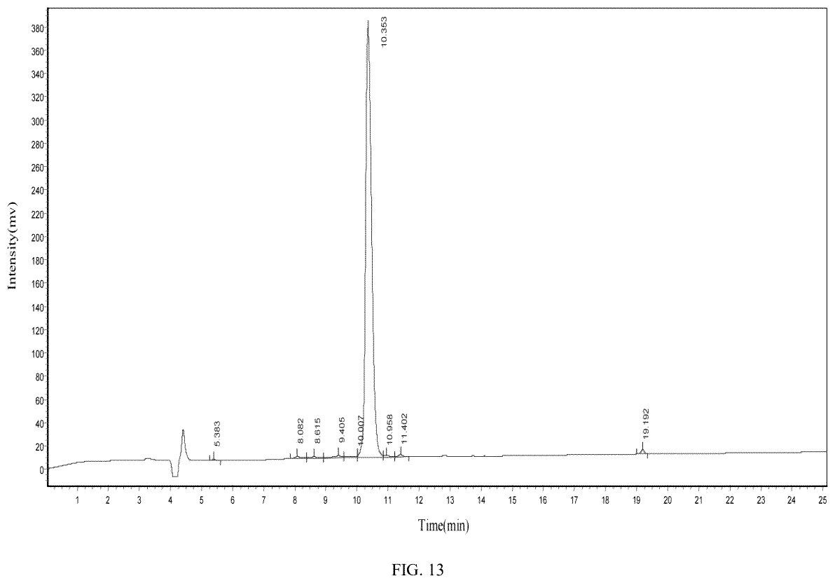

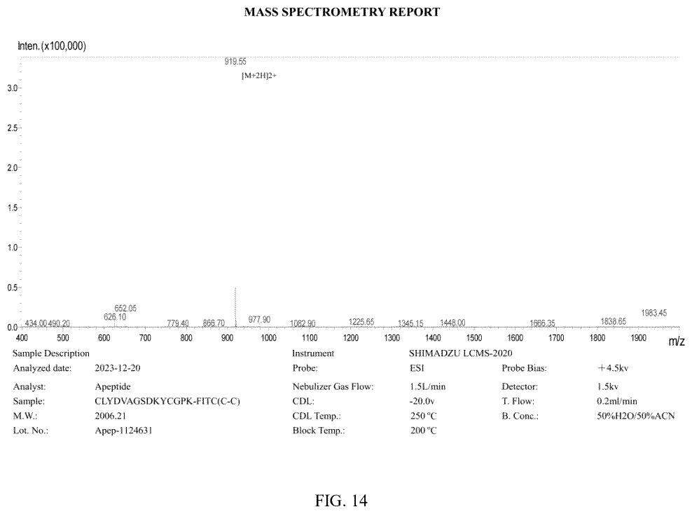

First, synthesize CLYDVAGSDKYCGPK fully protected linear peptide in the same manner as Embodiment 1, then cyclize the linear peptide, and finally couple the cyclic peptide with the fluorescent group FITC in the same manner as Embodiment 3 to obtain a fluorescently labeled cyclic peptide (HGP-FITC). Due to the lack of fluorescence of HGPF itself, HGP-FITC was used instead of HGPF for targeted testing of cyclic peptide conjugates on lung cancer cell lines. FIGS. 13 and 14 show the HPLC report and mass spectrometry of HGP-FITC, with the following structure:

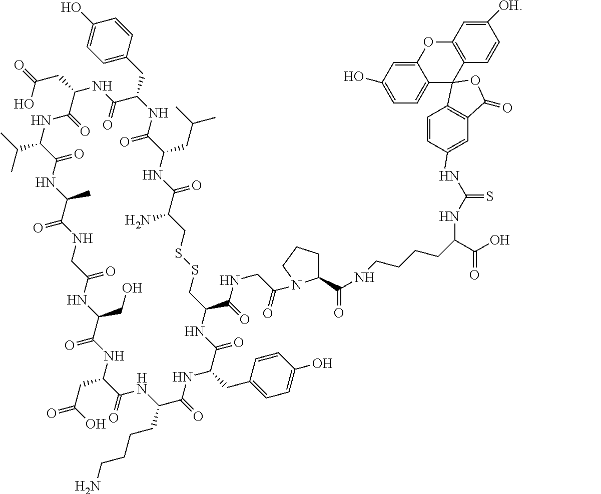 [SMILES: CC(C)C[C@@H]1NC(=O)[C@@H](N)CSSC[C@@H](C(=O)NCC(=O)N2CCC[C@H]2C(=O)NCCCCC(NC(=S)Nc2ccc3c(c2)C(=O)OC32c3ccc(O)cc3Oc3cc(O)ccc32)C(=O)O)NC(=O)[C@H](Cc2ccc(O)cc2)NC(=O)[C@H](CCCCN)NC(=O)[C@H](CC(=O)O)NC(=O)[C@H](CO)NC(=O)CNC(=O)[C@H](C)NC(=O)[C@H](C(C)C)NC(=O)[C@H](CC(=O)O)NC(=O)[C@H](Cc2ccc(O)cc2)NC1=O]

6.2 Incubate lung cancer cell line H358 (carrying KRAS G12C mutation) with different concentrations of HGP-FITC, and detect by flow cytometry after 8 hours.

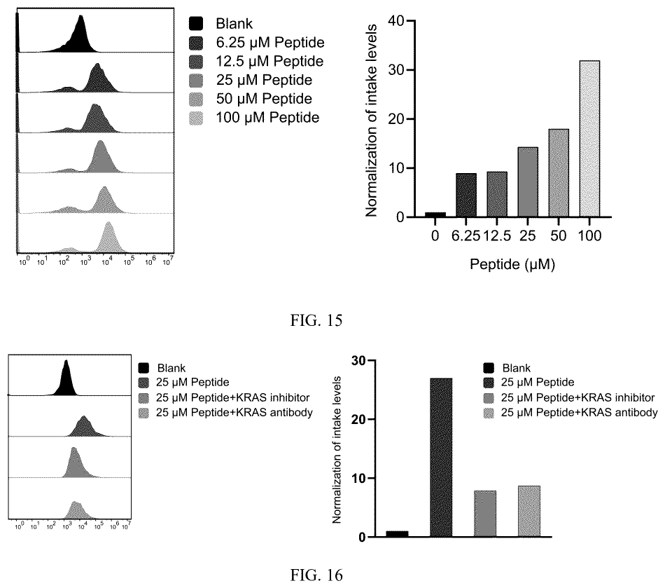

The flow cytometry results showed that H358 cells uptake HGP-FITC in a concentration dependent manner (See FIG. 15).

6.3 Based on the flow cytometry results, select a concentration of 25 μM for subsequent experiments. The lung cancer cell line H358 (carrying KRAS G12C mutation) was first blocked with 1:1000 KRAS antibody or 10 μM Sotorasib (KRAS G12C inhibitor) for 24 hours, and then incubated with 25 μM HGP-FITC. Fluorescence uptake was detected by flow cytometry after 8 hours.

The results showed that when KRAS antibody and Sotorasib were used to pre block the KRAS site, the uptake of HGP-FITC by H358 cells was significantly reduced (See FIG. 16). This result demonstrates that cyclic peptide conjugates such as HGP-FITC and HGPF have specific targeting ability towards KRAS G12C.

### Embodiment 7: FAPα Specific Recognition and Cutting Experiment for Polypeptides Containing GP Linkers

This embodiment uses a bioluminescence resonance energy transfer (BRET) experiment to demonstrate that the linker GP in HGPF can be specifically recognized and cleaved by FAPα. The principle is that when the distance between the energy donor RLUC fluorescent protein and the energy acceptor EYFP fluorescent protein is close, the emission wavelength of RLUC is 460 nm, which happens to be the excitation wavelength of EYFP, allowing EYFP to emit light at a wavelength of 530 nm. Firstly, construct a BRET reporter plasmid that can fuse and express EYFP, CLYDVAGSDKYCGP short peptide, and RLUC in cells. Simultaneously construct a plasmid overexpressing FAPα. If both plasmids are expressed simultaneously in mammalian cells, FAPα will cleave the fusion protein expressed in the reporter plasmid from the GP dipeptide, and energy transfer cannot occur after RLUC and EYFP are separated. Therefore, fluorescence detection at a specific wavelength can sensitively verify whether FAPα can cleave the linker. The specific steps are as follows:

### 7.1 Construction of BRET Report Plasmid:

### (1) Design Oligo Primers for Expressing CLYDVAGSDKYCGP Short Peptide:

F: ctgtctgtacgacgtcgcgggctccgataaatacggtcctgcaagcggttgtg (SEQ ID NO:1)
R: gatccacaaccgcttgcaggaccgtatttatcggagcccgcgacgtcgtacagacagagct (SEQ ID NO:2)

Two Oligo fragments were annealed to form double stranded DNA, which was then inserted into the BRET vector (purchased from Tsingke Biotech Co., Ltd.) through Sacl and BamHI enzyme cleavage sites to obtain the BRET reporter plasmid.

### 7.2 Construction of FAPα Overexpression Plasmid:

(1) The coding region sequence of FAPα gene is:

(SEQ ID NO: 3)
atgaagacttgggtaaaaatcgtatttggagttgccacctctgctgtgc
ttgccttattggtgatgtgcattgtcttacgcccttcaagagttcataa
ctctgaagaaaatacaatgagagcactcacactgaaggatattttaaat
ggaacattttcttataaaacattttttccaaactggatttcaggacaag
aatatcttcatcaatctgcagataacaatatagtactttataatattga
aacaggacaatcatataccattttgagtaatagaaccatgaaaagtgtg
aatgcttcaaattacggcttatcacctgatcggcaatttgtatatctag
aaagtgattattcaaagctttggagatactcttacacagcaacatatta
catctatgaccttagcaatggagaatttgtaagaggaaatgagcttcct
cgtccaattcagtatttatgctggtcgcctgttgggagtaaattagcat
atgtctatcaaaacaatatctatttgaaacaaagaccaggagatccacc
ttttcaaataacatttaatggaagagaaaataaaatatttaatggaatc
ccagactgggtttatgaagaggaaatgcttgctacaaaatatgctctct
ggtggtctcctaatggaaaatttttggcatatgcggaatttaatgatac
ggatataccagttattgcctattcctattatggcgatgaacaatatcct
agaacaataaatattccatacccaaaggctggagctaagaatcccgttg
ttcggatatttattatcgataccacttaccctgcgtatgtaggtcccca
ggaagtgcctgttccagcaatgatagcctcaagtgattattatttcagt
tggctcacgtgggttactgatgaacgagtatgtttgcagtggctaaaaa
gagtccagaatgtttcggtcctgtctatatgtgacttcagggaagactg
gcagacatgggattgtccaaagacccaggagcatatagaagaaagcaga
actggatgggctggtggattctttgtttcaacaccagttttcagctatg
atgccatttcgtactacaaaatatttagtgacaaggatggctacaaaca
tattcactatatcaaagacactgtggaaaatgctattcaaattacaagt
ggcaagtgggaggccataaatatattcagagtaacacaggattcactgt
tttattctagcaatgaatttgaagaataccctggaagaagaaacatcta
cagaattagcattggaagctatcctccaagcaagaagtgtgttacttgc
catctaaggaaagaaaggtgccaatattacacagcaagtttcagcgact
acgccaagtactatgcacttgtctgctacggcccaggcatccccatttc
cacccttcatgatggacgcactgatcaagaaattaaaatcctggaagaa
aacaaggaattggaaaatgctttgaaaaatatccagctgcctaaagagg
aaattaagaaacttgaagtagatgaaattactttatggtacaagatgat
tcttcctcctcaatttgacagatcaaagaagtatcccttgctaattcaa
gtgtatggtggtccctgcagtcagagtgtaaggtctgtatttgctgtta
attggatatcttatcttgcaagtaaggaagggatggtcattgccttggt
ggatggtcgaggaacagctttccaaggtgacaaactcctctatgcagtg
tatcgaaagctgggtgtttatgaagttgaagaccagattacagctgtca
gaaaattcatagaaatgggtttcattgatgaaaaaagaatagccatatg
gggctggtcctatggaggatacgtttcatcactggcccttgcatctgga
actggtcttttcaaatgtggtatagcagtggctccagtctccagctggg
aatattacgcgtctgtctacacagagagattcatgggtctcccaacaaa
ggatgataatcttgagcactataagaattcaactgtgatggcaagagca
gaatatttcagaaatgtagactatcttctcatccacggaacagcagatg
ataatgtgcactttcaaaactcagcacagattgctaaagctctggttaa
tgcacaagtggatttccaggcaatgtggtactctgaccagaaccacggc
ttatccggcctgtccacgaaccacttatacacccacatgacccacttcc
taaagcagtgtttctctttgtcagactaa

(2) The above sequence was inserted into the pcDNA3.1 (+) plasmid through two enzyme cleavage sites, BamHI and NotI, to obtain the FAPα overexpression plasmid.

### 7.3 Steps of BRET Experiment:

(1) Plate laying: Spread 293T cells in logarithmic growth phase evenly in a 6-well plate at a quantity of 3×105 per well, and culture for 12 hours in a 37° C., 5% CO2 cell culture incubator.

(2) Transfection: The FAPα overexpression plasmid and BRET reporter plasmid were transfected into 293T cells at a ratio of 9:1, while the FAPα and BRET reporter plasmids were separately transfected into 293T cells at the same amount as a control. After 48 hours, cells were lysed in each well of a 6-well plate using 200 μl of cell lysis buffer. The lysis buffer was collected and centrifuged to obtain the supernatant.

(3) Detection: First, prepare a 5 μM coelenterazine reaction solution with PBS, add 10 μl of cell lysis buffer to the enzyme-linked immunosorbent assay (ELISA) plate, followed by coelenterazine of 10 μl of 5 μM concentration, then incubate in the dark for 15 minutes, and then perform detection at wavelengths of 460 nm and 530 nm. The degree of BRET quantification is expressed as the ratio of emitted light at 460 nm to 530 nm.

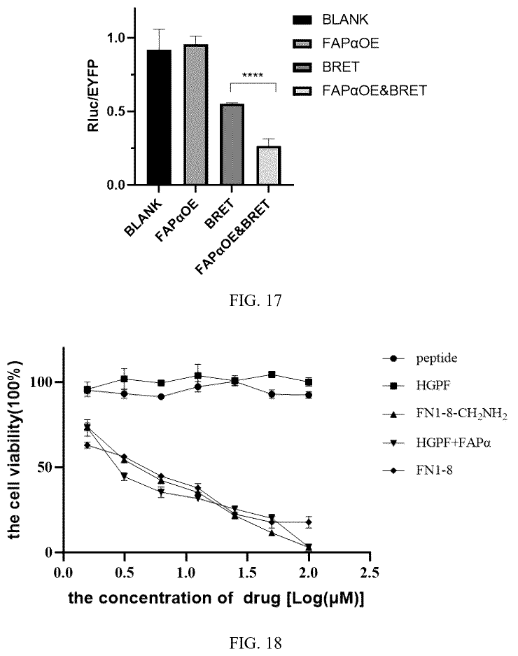

The results of the BRET experiment showed that GP short peptides can be specifically cleaved by FAPα (as shown in FIG. 17).

### Embodiment 8: Killing Effect of HGPF on Lung Cancer Cells

The cytotoxicity of HGPF, cyclic peptide (CLYDVAGSDKYCGP), FN1-8, and FN1-8 methylamino derivative (FN1-8-CH2-NH2) was detected through CCK8 experiment. The cyclic peptide (CLYDVAGSDKYCGP) has a sequence shown in SEQ ID NO: 4, and it is Cys-Leu-Tyr-Asp-Val-Ala-Gly-Ser-Asp Lys-Tyr-Cys-Gly-Pro. The experimental steps are as follows:

8.1 Plate laying: Inoculate H358 cells in logarithmic growth phase into a 96-well plate (100 μl/well), with 6 replicate wells per group and a cell count of 5×103 cells per well, then place and culture in a 37° C., 5% CO2 incubator for 12 hours.

8.2 Use different concentrations of HGPF, HGPF+FAPα, cyclic peptides, FN1-8, and FN1-8-CH2-NH2 to treat with the experimental group. First, prepare the above drugs into a 100 mM mother solution, and then set 9 concentrations, namely 100 μM, 50 μM, 25 μM, 12.5 μM, 6.25 μM, 3.125 μM, 1.5625 μM, and 0.78125 μM, wherein the HGPF+FAPα group was the group added FAPα (1 μM) after adding the above concentrations of HGPF to simulate the tumor tissue microenvironment.

8.3 After 24 hours, wash the cells twice with PBS, then add 100 μL of pre-mixed CCK8 solution (culture medium:CCK8 working solution=9:1) to each well, and then continue to culture in a 37° C., 5% CO2 incubator for 2 hours.

8.4 Use a microplate reader to measure the absorbance D (2) of each well at a wavelength of 450 nm. Analyze the D (2) values of the blank group, the control group, and the experimental group to determine cellular activity. The cell survival rate reflects the cytotoxicity of drugs. The groups are as follows:
Experimental group (culture medium containing cells, CCK-8, test drug)
Control group (culture medium containing cells, CCK-8, without test drug)
Blank group (culture medium without cells and test substance, containing CCK-8)

$$Cell survival rate = \frac{{D ( λ )}_{Experiment al group} - {D ( λ )}_{Blank group}}{{D ( λ )}_{Control group} - {D ( λ )}_{Blank group}} .$$

The CCK8 results (FIG. 18) showed that the cyclic peptide and HGPF groups without FAPα had weak cytotoxicity against lung cancer cells, while the HGPF group with FAPα, FN1-8 group, and FN1-8-CH2-NH2 group all had good cytotoxicity against tumor cells. Among them, the IC50 of FN1-8-CH2-NH2 is 4.676 μM, and the IC50 of FN1-8 is 4.341 μM, both of which have similar IC50 values. However, further increasing the concentration of FN1-8 does not significantly increase its cytotoxicity and cannot achieve the effect of completely inhibiting cell viability. When the concentration of FN1-8-CH2-NH2 is 100 μM, its inhibition rate on H358 cells can reach 100%, indicating that the killing effect of FN1-8-CH2-NH2 on tumor cells after structural modification of the present disclosure is improved compared to FN1-8. The IC50 of the conjugate HGPF is 3.656 μM, which is superior to the FN1-8-CH2-NH2 group, indicating that the HGPF conjugate obtained by coupling the cyclic peptide of the present disclosure with FN1-8-CH2-NH2 can improve the anti-tumor effect.

### Embodiment 9: Detection of the Inhibitory Effect of HGPF on Lung Cancer Cell Proliferation and Migration Using Scratch Test

The inhibitory effects of the conjugate HGPF and cyclic peptide (CLYDVAGSDKYCGP) on the proliferation and migration of lung cancer cells were detected by scratch test. The experimental steps are as follows:

9.1 Plate laying: First, mark 3 positioning lines at the bottom of the 6-well plate, then inoculate H358 cells in logarithmic growth phase onto the 6-well plate (2 ml/well), with a cell count of 1×10⁶ per well, and then culture in a 37° C., 5% CO2 incubator for 24 hours.

9.2 Marking: Place the plate cover above the 6-well plate, use a 100μ pipettor tip to draw 3 straight lines vertically for each hole; then wash 4 times with PBS to wash away floating cell debris.

9.3 Administration: Replace the culture medium with a medium containing 2% serum, administer 10 μM of the drug to each group, take photos at 0 and 24 hours, observe scratch healing, and analyze with ImageJ software.

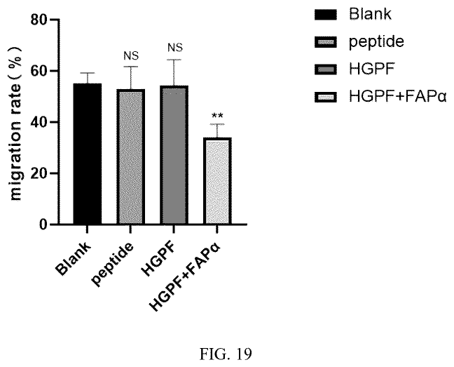

The scratch test results showed that the cyclic peptide and HGPF group without FAPα had no inhibitory effect on the proliferation and migration of tumor cells, while the HGPF group with FAPα significantly inhibited the proliferation and migration of tumor cells (FIG. 19).

The various technical features of the above embodiments can be combined arbitrarily. In order to make the description concise, not all possible combinations of the various technical features in the above embodiments have been described. However, as long as there is no contradiction in the combination of these technical features, they should be considered within the scope of this specification.

The above embodiments only show several embodiments of the present disclosure, with specific and detailed descriptions, but should not be understood as limiting the scope of the present disclosure patent. It should be pointed out that for ordinary skilled person in the art, several modifications and improvements can be made without departing from the inventive concept, which are within the scope of the present disclosure. Therefore, the scope of the present disclosure patent should be based on the appended claims.

1. A conjugate of a polypeptide and a small molecule, characterized in that, the conjugate is obtained by linking a cyclic peptide and a small molecule compound through a chemical bond;
wherein the structural formula of the cyclic peptide is: 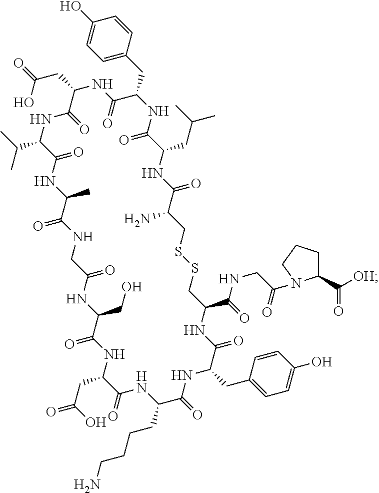 [SMILES: CC(C)C[C@@H]1NC(=O)[C@@H](N)CSSC[C@@H](C(=O)NCC(=O)N2CCC[C@H]2C(=O)O)NC(=O)[C@H](Cc2ccc(O)cc2)NC(=O)[C@H](CCCCN)NC(=O)[C@H](CC(=O)O)NC(=O)[C@H](CO)NC(=O)CNC(=O)[C@H](C)NC(=O)[C@H](C(C)C)NC(=O)[C@H](CC(=O)O)NC(=O)[C@H](Cc2ccc(O)cc2)NC1=O]
the small molecule compound is a small molecule inhibitor being capable of inhibiting GLI1 activity, and wherein the structural formula of the small molecule compound is:

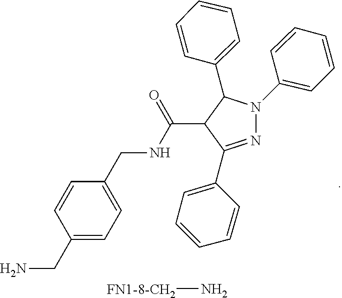

2. The conjugate of a polypeptide and a small molecule according to claim 1, wherein the carboxyl group at the C-terminal of the cyclic peptide is connected to the small molecule compound through an amide bond or an ester bond.

3. The conjugate of a polypeptide and a small molecule according to claim 1, wherein the structural formula of the conjugate is:

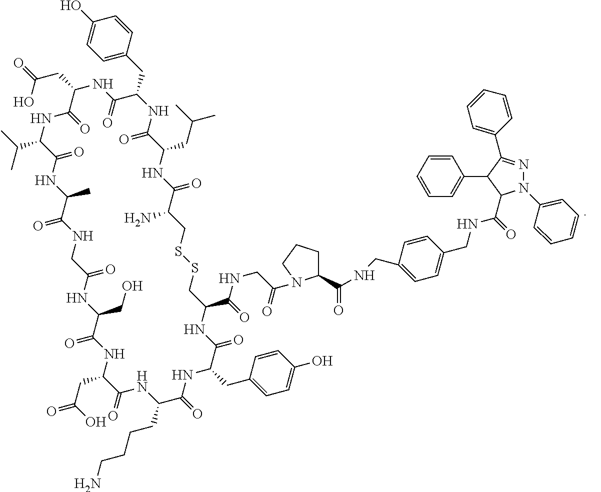 [SMILES: CC(C)C[C@@H]1NC(=O)[C@@H](N)CSSC[C@@H](C(=O)NCC(=O)N2CCC[C@H]2C(=O)NCc2ccc(CNC(=O)C3C(c4ccccc4)C(c4ccccc4)=NN3c3ccccc3)cc2)NC(=O)[C@H](Cc2ccc(O)cc2)NC(=O)[C@H](CCCCN)NC(=O)[C@H](CC(=O)O)NC(=O)[C@H](CO)NC(=O)CNC(=O)[C@H](C)NC(=O)[C@H](C(C)C)NC(=O)[C@H](CC(=O)O)NC(=O)[C@H](Cc2ccc(O)cc2)NC1=O]

4-8. (canceled)

9. A medicine for preventing and/or treating tumors, characterized in that, the medicine is prepared from an active ingredient and a pharmaceutically acceptable excipient, wherein the active ingredient comprises the conjugate of a polypeptide and a small molecule according to claim 1.

10-11. (canceled)

12. A medicine for preventing and/or treating tumors, characterized in that, the medicine is prepared from an active ingredient and a pharmaceutically acceptable excipient, wherein the active ingredient comprises the conjugate of a polypeptide and a small molecule according to claim 2.

13. A medicine for preventing and/or treating tumors, characterized in that, the medicine is prepared from an active ingredient and a pharmaceutically acceptable excipient, wherein the active ingredient comprises the conjugate of a polypeptide and a small molecule according to claim 3.
